(19) United States
(12) Patent Application Publication
(10) Pub. No.: US 2024/0033130 A1
(43) Pub. Date: Feb. 1, 2024

(54) PERFORATED TAPES FOR MEDICAL APPLICATIONS

(71) Applicant: 3M INNOVATIVE PROPERTIES COMPANY, St. Paul, MN (US)

(72) Inventors: Joel A. Getschel, Osceola, WI (US); Audrey A. Sherman, Woodbury, MN (US); Paul D. Graham, Woodbury, MN (US); David M. Mahli, Woodbury, MN (US)

(21) Appl. No.: 18/265,478

(22) PCT Filed: Dec. 9, 2021
(86) PCT No.: PCT/IB2021/061521

§ 371 Date: Jun. 6, 2023

Related U.S. Application Data
(60) Provisional application No. 63/124,167, filed on Dec. 11, 2020.

(51) Int. Cl. A61F 13/02

(52) U.S. Cl. CPC A61F 13/0269; A61F 13/0246

(57) ABSTRACT

The disclosure is directed to methods, systems, and apparatus for obtaining flame-perforated films which reduce or eliminate skewing of perforations in such films caused by thermal creep, whereby the film has perforations arranged to provide controlled tear characteristics, especially in both the lengthwise or machine direction (MD), and the crosswise or transverse direction (TD).

### BACKGROUND

The present disclosure is directed generally to forming films with perforations having controlled tearing characteristics. More particularly, the present disclosure is directed to obtaining flame-perforated films in a manner that eliminates or reduces the impact of thermal creep skewing perforations in the film, whereby the perforations have controlled tear characteristics in both the lengthwise or machine direction (MD), and the crosswise or transverse direction (TD).

Currently to obtain polymeric films with tear characteristics in the machine and crosswise directions, a simultaneously biaxially oriented polypropylene (SBOPP) film is utilized. A backing roll of a flame-perforating apparatus provides a supporting surface for the film as the latter is advanced through the apparatus. An exemplary flame-perforating apparatus is described in commonly assigned U.S. Pat. No. 7,037,100. The backing roll includes a plurality of lowered portions or etched wells formed in the backing roll surface. Each of the etched wells has a generally oval shape with a major axis oriented at 45 degree angles to crosswise or TD line of the advancing film web. Perforations are formed in the film over the etched wells as heat is applied to the advancing film by flames positioned over the etched wells. Collectively the noted wells are arranged in a generally herringbone pattern and, as such, it is expected that the resulting film perforations formed thereby would provide comparable tear characteristics in both the MD and TD directions. However, in practice, balanced tearing characteristics are relatively difficult to obtain. This is due to the impact of so-called thermal creep. Thermal creep as the term is used in the present application means the simultaneous application of heat and tension to the film during the flame-perforating process that results in the film undergoing thermal and physical stresses, such that the film stretches or elongates in the MD direction and shrinks or contracts in the TD dimension. As a result, the major axes of the resulting perforations are skewed in that they have angular orientations other than the 45 degrees intended to be imparted and other than the 45 degree orientation of the etched wells in the backing roll surface. As such, the tearing characteristics in both the MD and TD are unbalanced relative to their intended characteristics.

The condensation control process is one known approach for offsetting the impact of thermal creep causing skewing of the perforations particularly during a flame-perforating process. In particular, a film of water is generated on the backing roll while heat is applied by the flames. The resulting film of water causes adhesion between the film, preferably along the edges, and the backing roll. Adhesion inhibits the film slippage on the backing roll that arises, during the flame-perforating process, from the general simultaneous longitudinal expansion and transverse contraction of the film due to thermal creep. While condensation control has proven effective in minimizing the impact of thermal creep, such success has, however, been generally limited to situations involving relatively low tension forces being applied to the film or when low stresses from thermal creep are present. As a consequence, condensation control may not be as robust a process for large-scale commercial applications since significant tension forces must be applied to the larger and wider rolls of the film typically used commercially. The stresses imparted by thermal creep will also be larger in large-scale commercial equipment. In addition, the condensation control process requires utilization of control structures and methods for controlling the formation of the film of water, in order to provide successful implementations during the actual process. As such, this adds to overall commercialization costs and process complexity. Furthermore, because web tension forces are generally kept relatively low, any problems with uneven caliper in the input film cannot be overcome by increasing web tension.

Hence, needs exist for providing methods, systems, and apparatus for controlling tear characteristics of films, such as flame-perforated films. These needs further include being able to easily and reliably perforate film during a flame-perforating process, such that skewing of perforation orientations that are due to thermal creep are minimized or eliminated. These needs further include being able to provide tear characteristics wherein polymeric films, such as flame-perforated polymeric films, have comparable tear characteristics in both the lengthwise or machine direction (MD), and the crosswise or transverse direction (TD). These needs further include being able to correct for positional skewing of perforations in films, such as flame-perforated films, by thermal creep. These needs further include being able to, in a low cost manner, offset the impact of thermal creep skewing the orientations of perforations in the film. These needs further include being able to offset the impact of thermal creep skewing the orientation of perforations formed in the film in a manner that lessens the need for adhesion created by a water film, or the relatively expensive and complex water film control methods and mechanisms used during the actual process. The needs further include the ability to increase web tensions during the process so as to enable commercial processing of films requiring relatively high tension forces. Without such needs being satisfied the true potential for perforating films providing enhanced tear characteristics will not be fully achieved, especially in a simple, reliable, and less costly manner.

Accordingly, efforts are being undertaken for continuing the generation of improvements in this field that minimize the affects of thermal creep skewing the perforations in film during flame-perforating as well as being efficient and economical to implement.

### SUMMARY

In one exemplary embodiment, the present disclosure is directed to a method of correcting for positional skewing of perforations from a predefined angle of inclination relative to a generally transverse reference line of flame-perforated film produced by a flame-perforating process under a first set of conditions, the method comprising: determining the degree of angular deviation of the major axis of each of the one or more perforations in the flame-perforated film from the predefined angle of inclination; and forming one or more perforation-forming structures in a film supporting structure adapted for use in a subsequent flame-perforating process using the first set of conditions, wherein each of the perforation-forming structures has a major axis being angularly offset to the predefined angle of inclination by an inverse amount related to the angular deviation of the one or more corresponding perforations of the previously flame-perforated film.

In another exemplary embodiment, the present disclosure is directed to a film-supporting apparatus adapted to form perforations in film supported thereon during a flame-perforating process, wherein each of the formed perforations has a major axis positioned at a predefined angle of inclination relative to a generally transverse reference line, the apparatus comprises: a body having a film supporting surface, and one or more perforation-forming structures positioned on the film supporting surface, wherein each perforation-forming structure has a major axis angularly offset from the predefined angle of inclination of the major axis of each corresponding formed perforation by a predetermined amount.

In another exemplary embodiment, the present disclosure is directed to a roller adapted for use in a flame-perforating apparatus for perforating film, the roller comprises: a body; a film supporting surface on the body adapted to support and convey film to be perforated; and one or more perforation-forming structures on the supporting surface, each of which has a major axis having an angular orientation that is angularly offset to a predefined angle of inclination that is established relative to a generally transverse reference line across the film to be supported, wherein the angular offset is by an amount that is inversely related to the angular deviation relative to the predefined angle of inclination of one or more corresponding skewed perforations formed in previous flame-perforated film, the film supporting surface thus configured forms perforations in the film to be flame-perforated during a flame-perforating process that offsets the impact of thermal creep skewing the resulting one or more perforations, such that the major axis of each of the resulting one or more formed perforations is generally coincident with the predefined angle of inclination.

In another exemplary embodiment, the present disclosure is directed to a method of controlling tear characteristics of film, comprising: providing a polymeric film to be flame-perforated; providing a flame supporting apparatus that includes a body having a film supporting surface including one or more perforation-forming structures, each of the one or more perforation-forming structures has a major axis with an angular orientation that is angularly offset to a predefined angle of inclination established relative to a generally transverse reference line of film to be supported by the film supporting surface, wherein the angular offset is by an amount that is inversely related to the angular deviation relative to the predefined angle of inclination of one or more corresponding skewed perforations formed in previous flame-perforated film; and applying heat and tension forces to the film as it is advanced by the film supporting apparatus to form resulting perforations in the film; such that the film supporting surface thus configured forms perforations in the film supported thereby that offset the impact of thermal creep skewing the one or more resulting perforations, whereby the major axis of each of the resulting one or more perforations is generally coincident with the predefined angle of inclination.

In another exemplary embodiment, the present disclosure is directed to a method for use in an apparatus for flame-perforating a film, wherein the apparatus includes a film-supporting apparatus as noted above, the method of obtaining perforations during a flame-perforating process comprising: using the film-supporting apparatus as noted above during a flame-perforating process such that the formed perforations have a major axis at a predefined angle of inclination relative to a generally transverse reference line.

In another exemplary embodiment, the present disclosure is directed to a flame-perforated film made according to the above noted method of controlling tear characteristics in flame perforated film.

In another exemplary embodiment, the present disclosure is directed to a film comprising: first and second major surfaces; one or more perforations formed in at least one of the first and second major surfaces, wherein the one or more perforations in the film is formed by providing a film supporting apparatus that includes a film supporting surface having one or more perforation-forming structures thereon, each of the perforation-forming structures has a major axis with an angular orientation that is angularly offset to a predefined angle of inclination established relative to a generally transverse reference line of film to be supported by the film supporting surface, wherein the angular offset is by an amount that is inversely related to the angular deviation relative to the predefined angle of inclination of one or more corresponding skewed perforations formed in previous flame-perforated film; and applying heat and tension forces to the film as it is advanced by the film supporting apparatus such that the film supporting surface thus configured forms perforations in the film supported thereby during a flame-perforating process that offsets the impact of thermal creep skewing the resulting one or more perforations, such that the major axis of each of the resulting one or more formed perforations is generally coincident with the predefined angle of inclination to form perforations in the film.

In another exemplary embodiment, the present disclosure is directed to a flame-perforating apparatus for flame-perforating a film; the flame-perforating apparatus comprises: a frame; a first device coupled to the frame for applying heat to the film to form perforations in the film; and a second device coupled to the frame for advancing the film under tension through the apparatus, the second device includes a film supporting apparatus, the flame supporting apparatus includes a body having one or more perforation-forming structures on a film supporting surface thereof, each of the perforation-forming structures has a major axis with angular orientation angularly offset to a predefined angle of inclination established relative to a generally transverse reference line of film to be supported by the film supporting surface, wherein the angular offset is by an amount that is inversely related to the angular deviation relative to the predefined angle of inclination of one or more corresponding skewed perforations formed in previous flame-perforated film.

In another exemplary embodiment, the flame-perforating apparatus includes a water condensation control apparatus for controlling the formation of a film of water on the film supporting surface during the flame-perforating process.

In another exemplary embodiment, the present disclosure is directed to a system comprising: film comprising: first and second major surfaces; one or more perforations formed in at least one of the first and second major surfaces; and a flame-perforating apparatus for flame-perforating the film; the flame-perforating apparatus includes: a first device for applying heat to the film to form perforations in the film; and a second device for advancing the film under tension through the flame-perforating apparatus, the second device includes a film supporting apparatus, the film supporting apparatus includes one or more perforation-forming structures on a film supporting surface thereof, each of the perforation-forming structures has a major axis with an angular orientation that is angularly offset to a predefined angle of inclination established relative to a generally transverse reference line of film to be supported by the film supporting surface, wherein the angular offset is by an amount that is inversely related to the angular deviation relative to the predefined angle of inclination of one or more corresponding skewed perforations formed in previous flame-perforated film.

In another exemplary embodiment, the present disclosure is directed to an adhesive tape comprising: a flame-perforated film having first and second major surfaces as constructed above; a first film on one of the first and second major surfaces of the flame-perforated film; and a layer of adhesive coated on at least one of the first film and the other of the first and second major surfaces opposed to the surface having the first film thereon.

### Glossary

Thermal creep as the term is used in the present application means the simultaneous application of heat and tension to the film during the flame-perforating process that results in the film undergoing thermal and physical stresses, such that the film stretches or elongates in the MD direction and shrinks or contracts in the TD direction.

Perforation as the term is used in the present application means an opening made in or through something.

Transverse as the term is used in the present application is not limited to being perpendicular to an axis.

Skewing as the term is used in the present application to describe the perforations means that the major or longer axis of each of the perforations is at an angle that deviates from an intended angle.

Major axis as the term is used in the present application means a longitudinal axis of the larger of two axes of symmetry of a perforation or perforation-forming structure.

Angular offset as the term is used in the present application means the deviation between the actual major axis and the intended major axis.

Inverse amount as the term is used in the present application means an equal and opposite amount.

Perforation-forming structure as the term is used in the present application means any structure that results in the formation of a perforation in a flame-perforating process.

### BRIEF DESCRIPTION OF THE DRAWINGS

FIG. 1 is a side view of a flame-perforating of the present invention.

FIG. 2 is a front elevation view of the flame-perforating apparatus of FIG. 1 with two of the idler rolls and a motor removed for clarity and the backing roll shown in phantom lines.

FIG. 2A is an enlarged view of the ribbons of the burner of the apparatus as shown in FIG. 2.

FIG. 3 is a side view of the apparatus of FIG. 1 including the film along a film path in the apparatus.

FIG. 4 is an enlarged cross-sectional view of portions of the burner, film, and backing roll with a flame of the burner positioned away from the film, such that the flame is an unimpinged flame.

FIG. 5 is a view like FIG. 4 with the flame of the burner impinging the film.

FIG. 6 is a top plan view of a pattern of perforations in film, after the film has been perforated with the flame-perforating apparatus of FIG. 1.

FIG. 7 illustrates a cross-sectional view of a tape including film of the present invention.

FIG. 8 is an elevated view of a pattern of perforations in flame-perforated film.

FIG. 9 is a schematic view illustrating a portion of a flame-perforated film having perforations skewed relative to intended orientations.

FIG. 10 is a schematic view illustrating a portion of a film-supporting surface having perforation-forming structures therein with an angular orientation that is configured to provide a film during a flame-perforation process with perforations in an intended manner despite experiencing thermal creep.

FIG. 11 is a schematic view illustrating a portion of a flame-perforated film having perforations made following a flame-perforating process in which the film supporting surface of FIG. 10 has been used.

### DETAILED DESCRIPTION

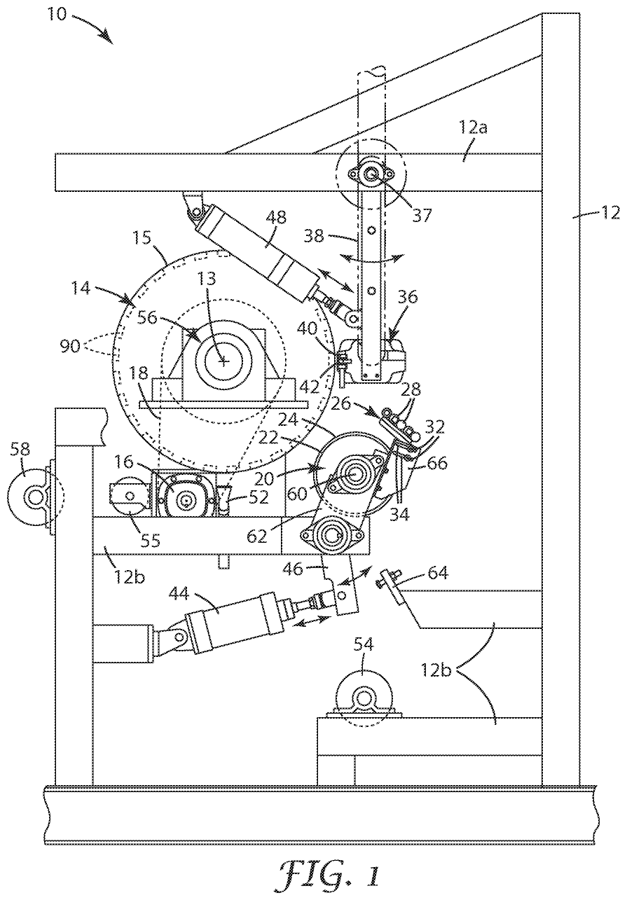

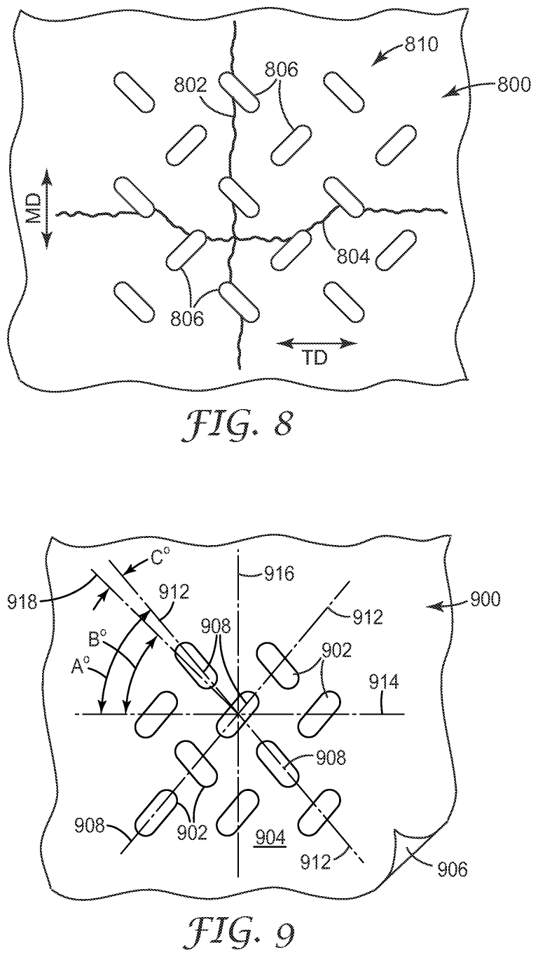

FIGS. 1-7 illustrate an apparatus, method and flame-perforated film that have perforations arranged in a herringbone pattern in order to provide comparable tear characteristics in both the lengthwise or machine direction (MD), and the crosswise or transverse direction (TD). FIGS. 1-7 are described in commonly assigned U.S. Pat. No. 7,037,100, which issued to the inventors of the present application which patent is incorporated herein in its entirety. It will be appreciated that those aspects of said patent which cooperate with the present invention will be described herein. In FIGS. 8-11, there is a description of a method, system, apparatus, and film, according to the present disclosure, which improve over the flame-perforating apparatus and process described in FIGS. 1-7. In particular, the improvements described in FIGS. 8-11 enable formation of flame-perforated film having comparable MD and TD tear characteristics in a manner that either does not require a water condensation process, and/or can be achieved on a commercial scale using high tension forces.

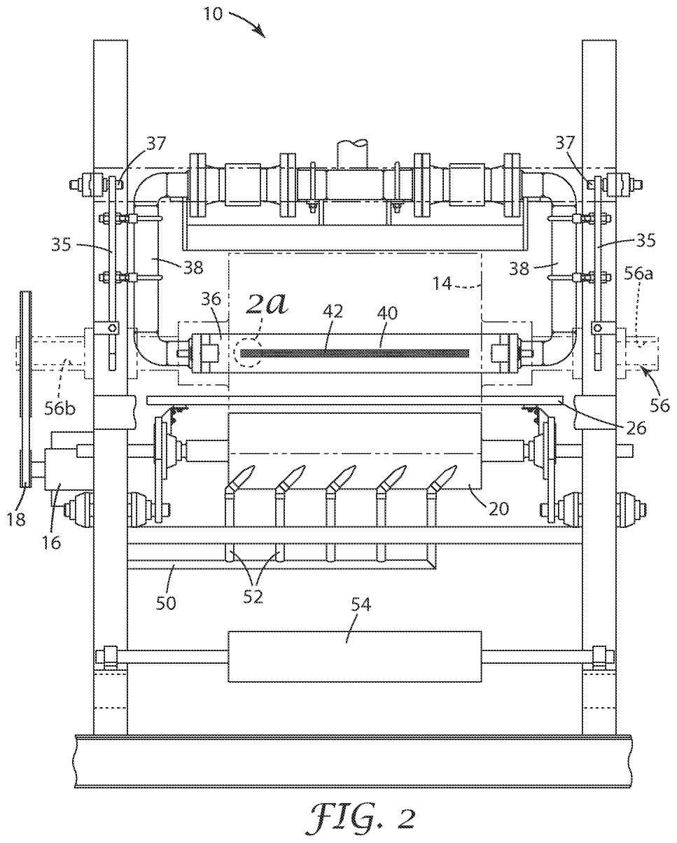

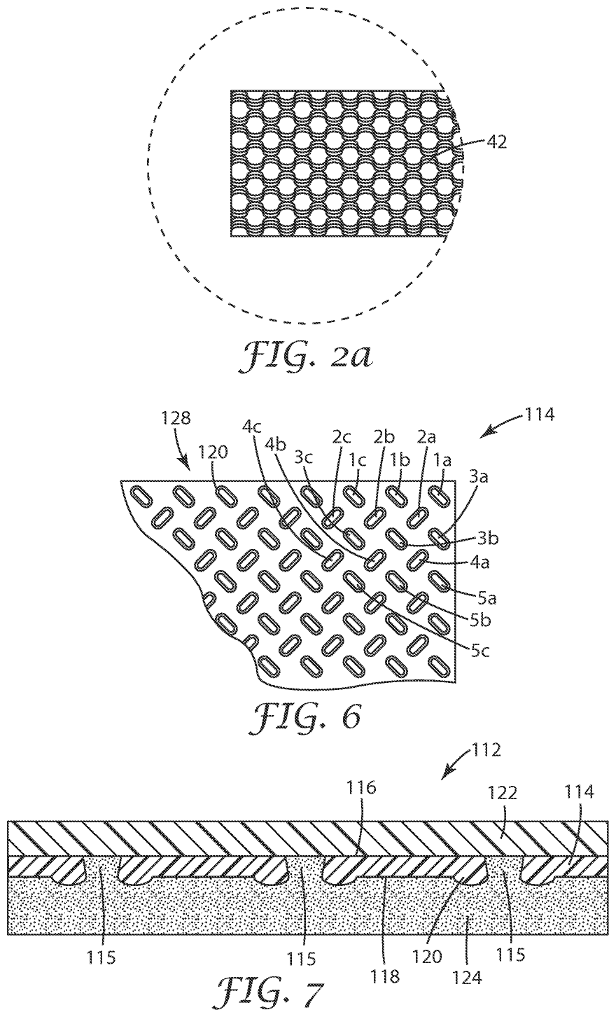

FIGS. 1 and 2 are illustrations of one preferred apparatus for making flame-perforated films according to the present invention. FIG. 1 illustrates a side view of the flame-perforating apparatus 10. FIG. 2 illustrates a front view of the flame-perforating apparatus with the backing roll 14 shown in phantom lines, and with the idler rollers 55, 38, and motor 16 removed, for clarity.

FIGS. 1 and 2 illustrate that the flame-perforating apparatus 10 includes a frame 12. The frame 12 includes an upper portion 12a and a lower portion 12b. The flame-perforating apparatus 10 includes a backing apparatus or roll 14 having an outer film support surface 15. The film support surface 15 typically includes a pattern of lowered portions 90, shown in phantom lines. These lowered portions 90 and the portions of the film support surface 15 between the lowered portions 90 collectively make up the film support surface 15 of the backing roll 14. The lowered portions 90 form a pattern of indentions in the film support surface 15. The lowered portions 90 may be a plurality of depressed or recessed portions or a plurality of indentations along the film support surface 15. These lowered portions 90 are typically etched into the film support surface 15. Alternatively, the pattern of lowered portions 90 may be drilled, ablated, or engraved into the film support surface 15. The lowered portions 90 typically are in the shape of ovals, and typically each have an approximate length of 70 mils (0.1778 cm) or less, an approximate width of 30 mils (0.0762 mm) or less, and an approximate depth of 8 mils (0.02032 cm) or more. One preferred example of a pattern of perforations is taught in PCT Publication, WO 02/11978, titled “Cloth-like Polymeric Films,” (Jackson et al.), which published on Feb. 14, 2002, which is hereby incorporated by reference.

Typically, the film support surface 15 of the backing roll 14 is temperature-controlled, relative to the ambient temperature around the flame-perforating apparatus 10. The film support surface 15 of the backing roll 14 may be temperature-controlled by any means known in the art. Typically, the film support surface 15 of the backing roll 14 is cooled by providing cooled water into the inlet portion 56a of hollow shaft 56, into the backing roll 14, and out of the outlet portion 56b of the hollow shaft 56. The backing roll 14 rotates about its axis 13. The flame-perforating apparatus 10 includes a motor 16 attached to the lower portion 12b of the frame. The motor 16 drives a belt 18, which in turn rotates the hollow shaft 56 attached to the backing roll 14, thus driving the backing roll about its axis 13.

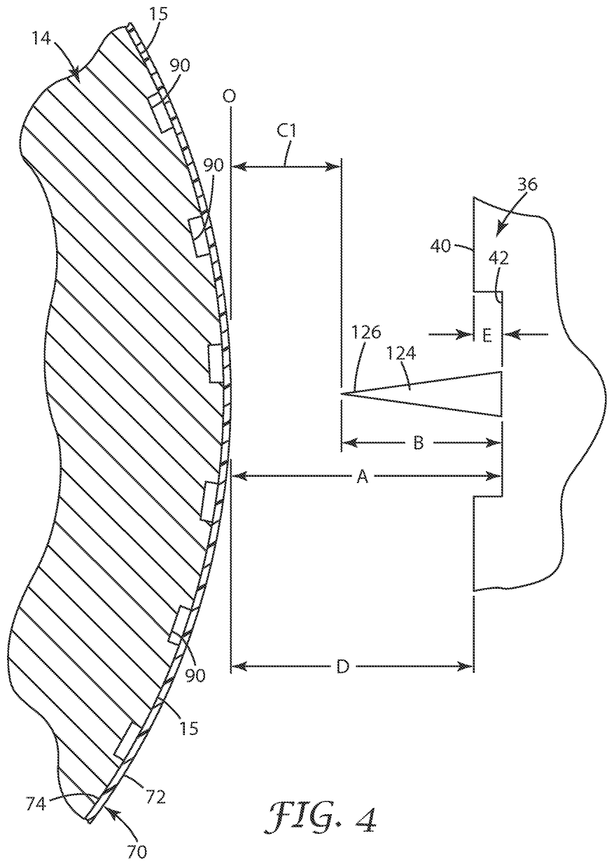

The flame-perforating apparatus 10 includes a burner 36 and its associated burner piping 38. The burner 36 and burner piping 38 are attached to the upper portion 12a of the frame 12 by burner supports 35. The burner supports 35 may pivot about pivot points 37 by actuator 48 to move the burner 36 relative to the film support surface 15 of the backing roll 14. The supports 35 may be pivoted by the actuator 48 to position the burner 36 a desired distance either adjacent or away from the film support surface 15 of the backing roll 14, as explained in more detail with respect to FIGS. 4 and 5 below. The burner 36 includes a gas pipe section 38 on each end for providing gas to the burner 36. The flame-perforating apparatus 10 may include an optional exhaust hood (not shown) mounted thereover.

In one exemplary embodiment of the present invention, the flame-perforating apparatus 10 includes a preheat roll 20 attached to the lower portion 12b of the frame 12. The preheat roll 20 includes an outer roll layer 22. The outer roll layer 22 includes an outer surface 24. Typically, the outer roll layer 22 is made of an elastomer; more typically, the outer roll layer is made of a high-service-temperature elastomer. Typically, the preheat roll 20 is a nip roll, which may be positioned against the backing roll 14 to nip the film between the nip roll 20 and backing roll 14. However, it is not necessary that the preheat roll 20 be a nip roll 20 and instead, the preheat roll may be positioned away from the backing roll 14 so as to not contact the backing roll 14. The nip roll 20 freely rotates about its shaft 60 and is mounted to roll supports 62. Linkage 46 is attached to roll supports 62. The nip roll 20 may be positioned against the backing roll 14, using actuator 44. When the actuator 44 is extended (as shown in FIG. 1), the linkage 46 is rotated counterclockwise, and in turn, the roll supports 62 are rotated counterclockwise until the nip roll 20 contacts the backing roll 14. The actuator 44 may control the movement between the nip roll 20 and the backing roll 14, and thus may control the pressure between the nip roll 20 and backing roll 14. A stop 64 is attached to the lower frame 12b to inhibit the movement of the linkage 46 beyond the lower frame 12b, which helps limit the pressure applied by the nip roll 20 against the backing roll 14.

In another embodiment, the flame-perforating apparatus 10 includes a temperature-controlled shield 26 attached to the nip roll 20 by brackets 66 to form one assembly. Accordingly, when the actuator 44 rotates the nip roll 20, as explained above, the temperature-controlled shield 26 moves with the nip roll. The temperature-controlled shield 26 may be positioned relative to the nip roll 20 by bolts 32 and slots 34 attached to the brackets 66. The temperature-controlled shield 26 typically includes a plurality of water-cooled pipes 28. However, other approaches of providing a temperature-controlled shield may be used, such as water-cooled plate, air-cooled plate, or other means in the art. Typically, the temperature-controlled shield 26 is positioned between the burner 36 and the nip roll 20. In this position, the shield 26 protects the nip roll 20 from some of the heat generated from the burner 36, and thus, can be used to control the temperature of the outer surface 24 of the nip roll 20, which has the benefits of reducing wrinkles or other defects in the film at the flame-perforating step performed by the burner 36, while maintaining high film speeds.

In yet another embodiment, the flame-perforating apparatus 10 includes an optional applicator 50 attached to the lower portion 12b of frame 12. The flame-perforating apparatus 10 includes a plurality of nozzles 52. In one exemplary embodiment, the applicator 50 is an air applicator for applying air onto the backing roll 14. In another embodiment, the applicator 50 is a liquid applicator for applying liquid onto the backing roll 14. Typically, the liquid is water; however, other liquids may be used instead. If the liquid is applied by the applicator 50, then typically, air is also supplied to the individual nozzles to atomize the liquid prior to application on the backing roll. The manner in which the air or water may be applied to the backing roll 14 may be varied by one skilled in the art, depending on the pressure, rate, or velocity of the air or water pumped through the nozzles 52. As explained below, without wishing to be bound by any theory, it is believed that if air or water is applied to the film support surface 15 of the backing roll 14, prior to contacting the film to the film support surface 15, then this application of air or water helps either remove some of the condensation built up on the film support surface 15 or applies additional water to actively control the amount of water between the film and the support surface, and thereby helps in eliminating wrinkles or other defects formed in the film at the flame-perforating step conducted by the burner 36.

The flame-perforating apparatus 10 includes a first idle roller 54, a second idle roller 55, and a third idle roller 58 attached to the lower portion 12b of the frame 12. Each idle roller 54, 55, 58 includes its own shaft and the idle rollers may freely rotate about their shafts.

FIG. 2A illustrates a blown-up view of the burner 36 useful with the apparatus 10 of FIG. 1. A variety of burners 36 are commercially available, for example, from the Flynn Burner Corporation, New Rochelle, NY; and Aerogen Company, Ltd., Alton, United Kingdom. One typical burner is commercially available from Flynn Burner Corporation as Series 860, which has an eight-port, 32 inch actual length that was deckled to 27 inch in length, stainless steel, deckled ribbon mounted in an extruded aluminum housing. A ribbon burner is most typically used for the flame perforation of polymer films, but other types of burners such as drilled-port or slot design burners may also be used. Typically, the apparatus includes a mixer to combine the oxidizer and fuel before it feeds the flame used in the flame-perforating process of the invention.

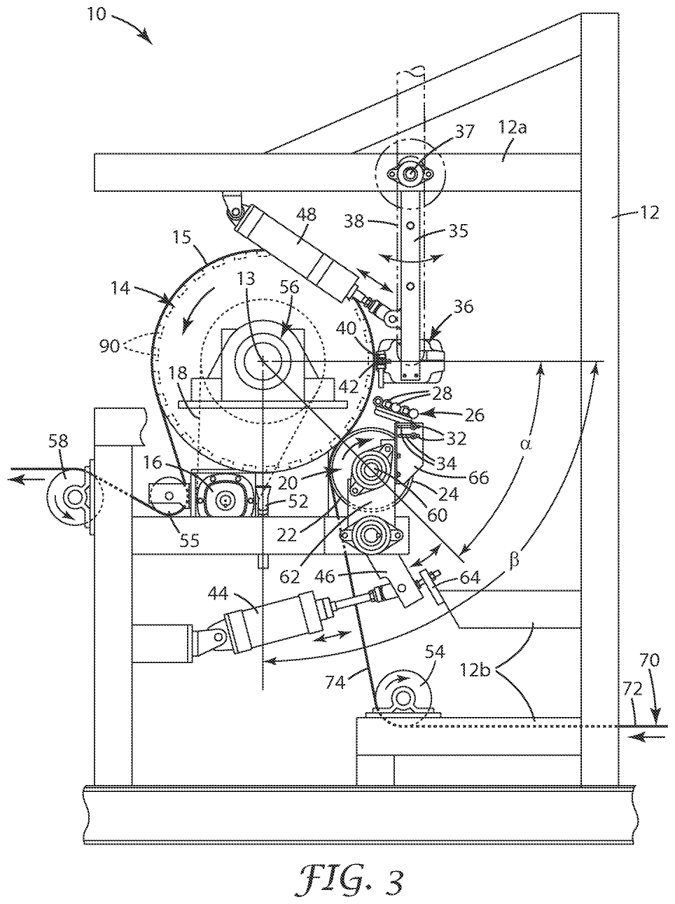

FIG. 3 illustrates the path that the film travels through the flame-perforating apparatus 10 and one exemplary method of flame-perforating films. The film 70 includes a first side 72 and a second side 74 opposite the first side 72. The film travels into the apparatus 10 and around the first idle roller 54. From there, the film is pulled by the motor-driven backing roll 14. In this position, the film is positioned between the nip roll and the backing roll 14. In this step of the process, the second side 74 of the film 70 is cooled by the water-chilled backing roll 14 and the first side 72 of the film 70 is simultaneously heated by the outer surface of the pre-heat or nip roll 20. This step of preheating the film 70 with the nip roll surface 22 of the nip roll 20 prior to flame-perforating the film with the burner 36 unexpectedly provided the benefits of reducing wrinkling or other defects in the film after the flame-perforating step was performed by the burner 36.

The temperature of the outer film support surface 15 of the backing roll 14 may be controlled by the temperature of the water flowing through the backing roll 14 through shaft 56. The temperature of the outer film support surface 15 may vary depending on its proximity to the burner 36, which generates a large amount of heat from its flames. In addition, the temperature of the film support surface 15 will depend on the material of the film support surface 15.

The temperature of the outer surface 24 of the outer layer 22 of the nip roll 20 is controlled by a number of factors. First, the temperature of the flames of the burner affects the outer surface 24 of the nip roll 20. Second, the distance between the burner 36 and the nip roll 20 affects the temperature of the outer surface 24. For example, positioning the nip roll 20 closer to the burner 36 will increase the temperature of the outer surface 24 of the nip roll 20. Conversely, positioning the nip roll farther away from the burner 36 will decrease the temperature of the outer surface 24 of the nip roll 20. The distance between the axis of nip roll 20 and the center of the burner face 40 of the burner 36, using the axis 13 of the backing roll 14 as the vertex of the angle, is represented by angle α. Angle α represents the portion of the circumference of the backing roll or the portion of the arc of the backing roll between the nip roll 20 and the burner 36. It is typical to make angle α, as small as possible, without subjecting the nip roll to such heat from the burner that the material on the outer surface of the nip roll starts to degrade. For example, angle α is typically less than or equal to 45°. Third, the temperature of the outer surface 24 of the nip roll 20 may also be controlled by adjusting the location of the temperature-controlled shield 26 between the nip roll 20 and the burner 36, using bolts 32 and slots 34 of the brackets 66. Fourth, the nip roll 20 may have cooled water flowing through the nip roll, similar to the backing roll 14 described above. In this embodiment, the temperature of water flowing through the nip roll may affect the surface temperature of the outer surface 24 of the nip roll 20. Fifth, the surface temperature of the film support surface 15 of the backing roll 14 may affect the surface temperature of the outer surface 24 of the nip roll 20. Lastly, the temperature of the outer surface 24 of the nip roll 20 may also by impacted by the ambient temperature of the air surrounding the nip roll 20.

Typical temperatures of the film support surface 15 of backing roll 14 are in the range of 45° F. to 130° F., and more typically are in the range of 50° F. to 105° F. Typical temperatures of the nip roll surface 24 of nip roll 20 are in the range of 165° F. to 400° F., and more typically are in the range of 180° F. to 250° F. However, the nip roll surface 24 should not rise above the temperature at which the nip roll surface material may start to melt or degrade. Although the temperatures of the support surface 15 of the backing roll 14 and the typical temperatures of the nip roll surface 24 of the nip roll 20 are listed above, one skilled in the art, based on the benefits of the teachings of this application, could select temperatures of the film support surface 15 and nip roll surface 24 depending on the film material and the rotational speed of the backing roll 14 to flame-perforate film with reduced numbers of wrinkles or defects.

Returning to the process step, at this location between the preheat roll 20 and backing roll 14, the preheat roll preheats the first side 72 of the film 70 prior to contacting the film with the flame of the burner. The temperature of the preheat roll 20 assists in eliminating wrinkles or other defects in the film at the flame-perforating step.

In the next step of the process, the backing roll 14 continues to rotate moving the film 70 between the burner 36 and the backing roll 14. This particular step is also illustrated in FIG. 5, as well as FIG. 3. When the film 70 comes in contact with the flames of the burner 36, the portions of the film that are directly supported by the chilled metal support surface are not perforated because the heat of the flame passes through the film material and is immediately conducted away from the film by the cold metal of the backing roll 14, due to the excellent heat conductivity of the metal. However, a pocket of air is trapped behind those portions of the film material that are covering the etched indentations or lowered portions 90 of the chilled support material. The heat conductivity of the air trapped in the indentation is much less than that of the surrounding metal and consequently the heat is not conducted away from the film. The portions of film that lie over the indentations then melt and are perforated. As a result, the perforations formed in the film 70 correlate generally to the shape of the lowered portions 90. At about the same time that film material is melted in the areas of the lowered portions 90, a raised ridge or edge 120 is formed around each perforation, which consists of the film material from the interior of the perforation that has contracted upon heating.

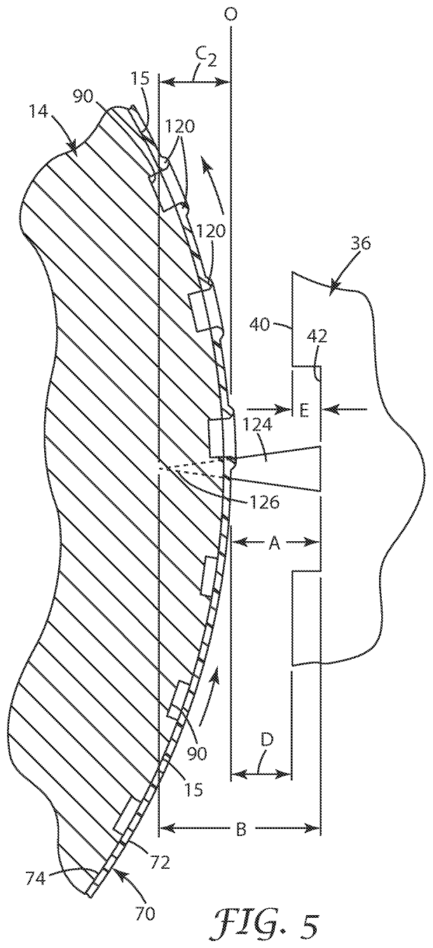

After the burner 36 has flame-perforated the film, the backing roll 14 continues to rotate, until the film 70 is eventually pulled away from the film support surface 15 of the backing roll 14 by the idler roller 55. From there, the flame-perforated film 70 is pulled around idler roll 58 by another driven roller (not shown). The flame-perforated film may be produced by the flame-perforating apparatus 10 in long, wide webs that can be wound up as rolls for convenient storage and shipment. Alternatively, the film 70 may be combined with a layer of pressure-sensitive adhesive or other films to provide tape, as discussed in reference to FIG. 7.

As mentioned above, the flame-perforating apparatus 10 may include the optional applicator 50 for either applying air or water to the film support surface 15 of the backing roll 14, prior to the film 70 contacting the support surface between the backing roll 14 and the nip roll 20. Without wishing to be bound by any theory, it is believed that controlling the amount of water between the film 70 and the film support surface 15 helps reduce the amount of wrinkles or other defects in the flame-perforated film. There are two ways in which to control the amount of water between the film 70 and the film support surface 15. First, if the applicator 50 blows air onto the support surface, then this action helps reduce the amount of water build up between the film 70 and film support surface 15. The water build up is a result of the condensation formed on the backing roll surface when the water-cooled film support surface 15 is in contact with the surrounding environment. Second, the applicator 50 may apply water or some other liquid to the film support surface 15 to increase the amount of liquid between the film 70 and the support surface. Either way, it is believed that some amount of liquid between the film 70 and the film support surface 15 may help increase the fraction between the film 70 and the film support surface 15, which in turn helps reduce the amount of wrinkles or other defects in the flame-perforated film. The position of the nozzles 52 of the applicator 50 relative to the centerline of the burner 36 is represented by angle β where the vertex of the angle is at the axis 13 of the backing roll 14. Typically, the applicator 50 is at an angle β greater than angle α so that the air or water is applied to the backing roll 14 prior to the nip roll 20.

Maintaining some level of water in between the backing roll and the film improves overall quality of the perforated film. However, it was also observed that poor perforation quality would also result with an excess of water applied to the indentation pattern of the backing roll because water that is either partially or completely filling the indentations provides such good heat conductivity that the film over the indentations is not exposed to sufficient heat to form perforations in the film.

FIGS. 4 and 5 schematically illustrate yet another embodiment of the flame-perforating apparatus of the present invention. FIGS. 4 and 5 illustrate the placement of the flame 124 relative to the film support surface 15 of the backing roll 14 during the flame-perforating step. In FIG. 4, the burner 36 is at some distance relative to the backing roll 14, and in FIG. 5, the burner 36 is positioned closer to the backing roll 14 relative to FIG. 4. The relative distance between the burner 36 and backing roll 14 may be adjusted by the burner supports 35 and the actuator 48, as explained above in reference to FIG. 1.

There are several distances represented by reference letters in FIGS. 4 and 5. Origin “O” is measured at a tangent line relative to the first side 72 of the film wrapped around the backing roll 14. Distance “A” represents the distance between the ribbons 42 of the burner 40 and the first side 72 of the film 70. Distance “B” represents the length of the flame, as measured from the ribbons 42 of the burner 36, where the flame originates, to the tip 126 of the flame. The flame is a luminous cone supported by the burner, which can be measured from origin to tip with means known in the art. Actually, the ribbon burner 36 has a plurality of flames and typically, all tips are at the same position relative to the burner housing, typically uniform in length. However, the flame tips could vary, for example, depending on non-uniform ribbon configurations or non-uniform gas flow into the ribbons. For illustration purposes, the plurality of flames is represented by the one flame 124. Distance “D” represents the distance between the face 40 of the burner 36 and the first side 72 of the film 70. Distance “E” represents the distance between the ribbons 42 of the burner 36 and the face 30 of the burner 36.

In FIG. 4, distance “C1” represents the relative distance between distance A and distance B, if they were subtracted A-B. This distance C1 will be a positive distance because the flame 124 is positioned away from the backing roll 14 and thus, does not impinge the film 70 on the backing roll 14, and is defined as an “unimpinged flame.” In this position, the flame may be easily measured in free space by one skilled in the art, and is an uninterrupted flame. In contrast, FIG. 5 illustrates the burner positioned much closer to the film 70 on the backing roll 14, such that the tip 126 of the flame 124 actually impinges the film 70 on the film support surface 15 of the backing roll 14. In this position, “C2” represents distance A subtracted from distance B, and will necessarily be a negative number. Typically, distance A subtracted from distance B is greater than a negative 2 mm. Unexpectedly, it was found that perforated films could be produced at higher speeds with a C2 distance of large negative numbers, while still maintaining film quality. This was unexpected in light of the prior art, which teaches that optimal flame conditions are achieved with a positive or zero C1 distance.

Typically, the film 70 is a polymeric substrate. The polymeric substrate may be of any shape that permits perforation by flame and include, for example, films, sheets, porous materials and foams. Such polymeric substrates include, for example, polyolefins, such as polyethylene, polypropylene, polybutylene, polymethylpentene; mixtures of polyolefin polymers and copolymers of olefins; polyolefin copolymers containing olefin segments such as poly(ethylene vinylacetate), poly(ethylene methacrylate) and poly(ethylene acrylic acid); polyesters, such as poly(ethylene terephthalate), poly(butylene phthalate) and poly(ethylene naphthalate); polystyrenes; vinylics such as poly(vinyl chloride), poly(vinylidene dichloride), poly(vinyl alcohol) and poly(vinyl butyral); ether oxide polymers such as poly(ethylene oxide) and poly(methylene oxide); ketone polymers such as polyetheretherketone; polyimides; mixtures thereof, or copolymers thereof. For example, the polymeric material is from a group that comprises simultaneously or sequentially biaxially oriented polypropylene film and uniaxially oriented polypropylene film. Typically, the film is made of oriented polymers and more typically, the film is made of biaxially oriented polymers. Biaxially oriented polypropylene (BOPP) is commercially available from several suppliers including: ExxonMobil Chemical Company of Houston, Tex.; Continental Polymers of Swindon, UK; Kaisers International Corporation of Taipei City, Taiwan and PT Indopoly Swakarsa Industry (ISI) of Jakarta, Indonesia. Other examples of suitable film material are taught in the aforenoted PCT Publication, WO 02/11978, titled “Cloth-like Polymeric Films,” (Jackson et al.).

FIG. 6 illustrates a top view of a pattern of perforations in film after it has been perforated with the flame-perforating apparatus of FIG. 1. The perforations are typically elongate ovals, rectangles, or other non-circular or circular shapes arranged in a fashion such that the major axis of each perforation intersects adjacent perforations or passes near adjacent perforations. This perforated polymeric film 114 can be joined to one or more additional layers or films, such as a top layer to provide durability or impermeability, or a bottom layer to provide adhesiveness.

The perforation pattern formed in polymeric film 114 has a strong influence on the tear and tensile characteristics of the perforated films and tape backings of the invention. In FIG. 6, a portion of an enlarged layout of a typical perforation pattern 128 is shown, with the machine direction oriented up and down, and the transverse direction oriented left to right. Depicted perforation pattern 128 comprises a series of rows of perforations, identified as a first row having perforations 1a, 1b, and 1c; a second row having perforations 2a, 2b, and 2c; a third row having perforations 3a, 3b, and 3c; a fourth row having perforations 4a, 4b, and 4c; and a fifth row having-perorations 5a, 5b, and 5c. The perforation pattern 128 includes other rows of perforations, similar to the first row through the fifth row. Each perforation includes a raised ridge or edge 120. In specific implementations, this raised ridge 120 has been observed to provide enhanced tear characteristics of the perforated film 114. The raised ridge 120 can also impart slight textures that cause the film 114 to more closely resemble a cloth-like material. Typically the perforations form a pattern extending along most or all of the surface of a film, and thus the pattern shown in FIG. 6 is just a portion of one such pattern.

As explained above in reference to FIG. 5, the perforation pattern 128 formed in the film 114 correlates generally to the pattern of lowered portions 90 formed into the film support surface 15 of the backing roll 14. The film shown in FIG. 6 includes numerous perforations, each of which is generally oval-shaped, and typically includes a length of approximately two or three-times greater than the width. However, one skilled in the art could select any pattern of lowered portions 90 in film support surface 15 of the backing roll 14 to create alternative perforation patterns or sizes.

The films described herein are suited for many adhesive tape backing applications. The presence of a top film over the perforation pattern can provide an appearance similar to a poly-coated cloth-based tape backing in certain embodiments. This appearance, combined with the tensile and tear properties, makes the film useful as a backing for duct tape, gaffer's tape, or the like. Because the backing is conformable, it is also useful as a masking tape backing.

FIG. 7 illustrates a cross-sectional view of one embodiment of a tape 112 including the film of FIG. 6 as a tape backing. Tape 112 contains a perforated film 114 having a first major surface 116 and a second major surface 118. Perforated film 114 contains perforations 115 extending through its thickness. In the embodiment illustrated, the edges of each perforation 115 along second major surface 118 include raised portions 120. Perforated film 114 is typically an oriented film, more typically a biaxially oriented polypropylene film.

Polymeric tape 112 further includes a top film 122 and a bottom layer 124. In the embodiment illustrated, top film 122 provides durability to the polymeric tape 112, and can further increase the strength and impart fluid impermeability to tape 112. Bottom layer 124 is, for example, an adhesive composition. Additional or alternative layers can be used to create tape 112. The arrangement of the layers can also be changed. Thus, for example, the adhesive can be applied directly to the top film 122 rather than to the perforated film 114.

The operation of the apparatus 10 will be further described with regard to the following detailed examples. These examples are offered to further illustrate the various embodiments and techniques. It should be understood, however, that many variations and modifications may be made while remaining within the scope of the application.

The custom-designed flame perforation system described above was used to generate the examples below, wherein the perforated film is made of biaxially oriented polypropylene (BOPP). Dust-filtered, 25° C. compressed air was premixed with a natural gas fuel (having a specific gravity of 0.577, a stoichiometric ratio of dry air:natural gas of 9.6:1, and a heat content of 37.7 kJ/L) in a venturi mixer, available from Flynn Burner Corporation, of New Rochelle, NY., to form a combustible mixture. The flows of the air and natural gas were measured with mass flow meters available from Flow Technology Inc. of Phoenix, AZ. The flow rates of natural gas and air were controlled with control valves available from Foxboro-Eckerd. All flows were adjusted to result in a flame equivalence ratio of 0.96 (air:fuel ratio of 10:1) and a normalized flame power of 20,000 Btu/hr-in. (2135 W/cm²). The combustible mixture passed through a 3 meter long pipe to a ribbon burner, which consisted of a 68 cm×1 cm, 8-port corrugated stainless steel ribbon mounted in an extruded aluminum housing, supplied by Flynn Burner Corporation, New Rochelle, NY.

The burner was mounted adjacent a 61 cm diameter, 76 cm face-width, steel, spirally-wound, double-shelled, chilled backing roll, available from F. R. Gross Company, Inc., Stow Ohio. The temperature of the backing roll was controlled by a 240 l/min recirculating flow of water at a temperature of 50° F. (10° C.). The steel backing roll core was plated with 0.5 mm of copper of a 220 Vickers hardness, and then engraved by Custom Etch Rolls Inc. of New Castle, PA, with a perforation pattern shown in FIG. 6.

An electric spark ignited the combustible mixture. Stable conical flames were formed with tips approximately 7 mm from the face of the burner housing. The ribbons were recessed 3 mm from the face of the burner. A thermally extruded, biaxially oriented polypropylene (BOPP) homopolymer film, which was 1.2 mil (0.03 mm) thick and 68.5 cm wide, was guided by idler rolls to wrap around the chilled backing roll and processed through the system at an adjustable speed. The upstream tension of the film web was maintained at approximately 2.2 N/cm and the downstream tension was approximately 2.6 N/cm.

To insure intimate contact between the BOPP film and the chilled backing roll, a 23 cm diameter, 76 cm face-width, inbound nip roll, available from American Roller Company, Kansasville, WI, covered with 6 mm of VN 110 (80 Shore A durometer) VITON fluoroelastomer, was located at an adjustable position of approximately 45 degrees relative to the burner, on the inbound side of the chilled backing roll. A water-cooled shield was positioned between the nip roll and the burner which was maintained at a temperature of 50° F. (10° C.) with recirculating water. The nip roll-to-backing roll contact pressure was maintained at approximately 50 N/lineal cm. The film speed through the flame perforation system was 91 m/min.

A custom-built air impingement system utilizing 6 air nozzles was installed to blow compressed air onto the chilled backing roll at a pressure of 10 PSI (69 kPa/m 2) to controllably reduce the amount of water condensation accumulating on the patterned portion of the backing roll. The air nozzles were located approximately 45 degrees prior to the nip roll, relative to the axis of the backing roll.

FIG. 8 is another view that is representative of a polymeric flame-perforated film 800 that is formed by the flame-perforating process described above, but illustrating MD and TD tear lines 802 and 804; respectively. The flame-perforated film 800 includes numerous perforations 806, each of which is generally oval in shape and has a length with a major axis that is greater than a minor axis across the width. Raised ridges like those described above in FIG. 6 that normally surround each of the perforations 806 in a flame-perforating process have not been illustrated for purposes of clarity. Rows and columns of the perforations 806 are oriented at angles of approximately 45 degrees to the lengthwise or machine direction (MD) and the crosswise or transverse direction (TD) in order to obtain comparable tearing in both the MD and TD. Adjacent rows of perforations are oriented at opposed angles and form essentially a so-called herringbone pattern 810. This herringbone perforation pattern 810 is configured and arranged in a manner such that the polymeric film is intended to possess tear characteristics that provide both a relatively straight MD tear line 802 and TD tear line 804. An example of such a film perforation pattern is described in the aforenoted PCT Application, entitled “Cloth-like Polymeric Films”.

FIG. 9 illustrates a flame-perforated film 900 that has its perforations 902 skewed relative to their intended orientations, such as illustrated in FIGS. 6 and 8, as will be explained. The perforations 902 may extend through two opposed major surfaces 904, 906. Raised ridges like those described above in FIG. 6 that normally surround each of the perforations 902 in a flame-perforating process have not been illustrated for purposes of clarity. In the illustrated embodiment, each one of the perforations 902 includes a major axis 908. While symmetrical perforations are illustrated, non-symmetrical perforations may be used.

As noted, the perforations 902 have their orientations skewed relative to the orientations of the perforations in the films depicted in FIGS. 6 and 8. For example, the films described in FIGS. 6 and 8 had their perforations at a predefined 45 degrees to a transverse reference line across the web of the film. In contrast, as a result of thermal creep, the perforations 902 are skewed or deviate from the predefined 45 degrees, such that they are at about 51 degrees with respect to transverse reference line. This is an increase of about 6 degrees from the intended orientation of 45 degrees.

An undesirable aspect of skewing is that it alters the tearing characteristics desired to be imparted by the pattern and orientations of the perforations. Because of skewing comparable tear characteristics in the MD and TD are diminished. Skewing, as noted, results from thermal creep. As noted, the foregoing process set forth in FIGS. 1-7 allows thermal creep to be introduced in several ways. In this latter regard, skewing of the perforations 902 may be accentuated by a set of conditions in the flame-perforating process typically used for producing film commercially wherein higher web tension forces are used than with film made according to U.S. Pat. No. 7,037,100 patent. In typical commercial processing, the set of conditions includes at least higher tension forces which exceed the ability of the water condensation process, such as described in U.S. Pat. No. 7,037,100, preventing the perforations from skewing.

Each of the perforations 902 has its major axis 908 coincident with an illustrated perforation skew line 912. The perforation skew line 912 defines an angle A with a generally transverse reference line 914. The perforation skew line assumed by the major axis of the perforation is offset relative to its intended angular orientation. In an exemplary embodiment, angle A is 51 degrees.

The transverse reference line 914 need not be perpendicular to a longitudinal axis 916 of an advancing film being supported by a supporting backing roll (not shown). In this embodiment, however, the transverse reference line 914 is generally coincident to the transverse direction (TD) of the film and is perpendicular the longitudinal axis 916 as well. Transverse reference lines having angles other than 90 degrees to the longitudinal axis 916 are contemplated. The perforation skew line 912 has an angular deviation relative to a predefined angle of inclination illustrated by reference line 918. The predefined angle of inclination line 918 is measured relative to a same transverse reference line 914. The predefined angle of inclination line 918 defines an angle B relative to the transverse reference line 914. The predefined angle of inclination line 918 is the line that is intended to be coincident to the intended angle the major axis of each perforation has with respect to the generally transverse reference line 914. As noted, such a relationship will enable the perforations to impart the desired tearing characteristics. In the exemplary embodiment, the angle B is 45 degrees and assists in obtaining comparable tear characteristics in the MD and TD. An angle C of deviation is provided that represents the angular deviation of the skew line 912 including the major axis 908 of a perforation with respect to the predefined angle of inclination 918. The angle of deviation (angle C) is directly attributable to the thermal creep and represents the angular amount of deviation of the perforations 902.

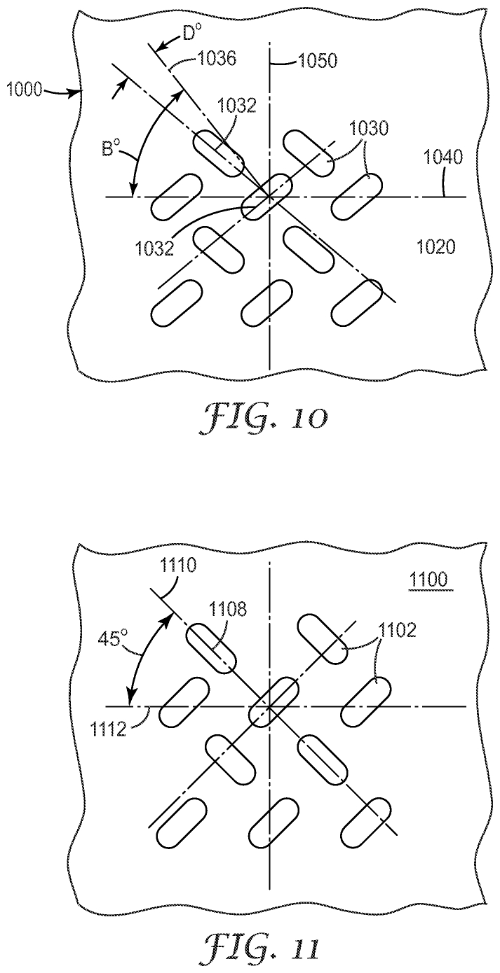

Reference is made to FIG. 10 for illustrating a portion of a film supporting apparatus 1000 that is adapted to reduce or eliminate the effects of thermal creep skewing perforations on a flame-perforated film 1010, such as perforations 902 on the flame-perforated film 900.

The film supporting apparatus 1000 includes a film supporting surface 1020 that is adapted to support and convey the film (not shown) through the flame-perforation apparatus 10. In one exemplary embodiment, the film supporting apparatus 1000 is implemented as a backing roll 1000. The backing roll 1000 may have a 610 mm diameter, with a 760 mm face width. The backing roll 1000 may be a water-cooled steel backing roll for flame-perforation. Such a surface may be polished to a finish suitable for etching of one or more perforation-forming structures 1030 therein. The one or more perforation-forming structures 1030 can be arranged with a pattern as will be described so as to reduce or eliminate skewing caused by thermal creep.

Essentially, the present disclosure is directed to a method of correcting for positional skewing of perforations, such as illustrated in FIG. 9 from their predefined angle of inclination relative to a generally transverse reference line across the flame-perforated film produced by a flame-perforating process under a first set of conditions. The set of perforation process conditions are similar to those described earlier.

It will be appreciated that the corrections that are to be introduced by offsetting the perforation-forming structures 1030, in a manner to be described, are effective so long as the set of flame-perforating process conditions that caused the skewing in the first place are the same or are a similar set of conditions that will be used in subsequent flame-perforating process with the improved film supporting apparatus 1000. In other words, the improved film supporting apparatus 1000 may not obtain the desired perforation offsetting in subsequent flame-perforating steps even if operating in the flame-perforating apparatus, should the operating conditions which caused the skewing in the first instance be changed significantly.

The method of this disclosure comprises determining the degree of angular deviation (i.e., angle C), see FIG. 9, of the major axis of each of the one or more perforations skewed from the predefined angle of inclination (i.e., angle B). Stated differently an operator will determine through suitable techniques the angular deviation angle C of perforations, as noted above in film 900.

To correct for the skewing in film 900 according to the present disclosure, the operator then forms a corresponding one or more perforation-forming structures 1030 in the backing roll 1000 (FIG. 10) so that each has its major axis 1032 angularly offset to the predefined angle of inclination (angle B) as represented by the line 1036 by an offset amount (i.e., angle D of FIG. 10). The predefined angle of inclination is related to a transverse reference line 1040 that may be perpendicular to the longitudinal axis 1050. The offset angle (i.e., angle D) is inversely related to the angular deviation (i.e., angle C of FIG. 9) of the one or more corresponding perforations 902 of the previously flame-perforated film. By inversely related it is meant that if the angle of deviation (i.e., angle C) calculated from FIG. 9 is greater or lesser than the predefined angle of inclination (i.e., angle B) then the angle of the major axis of the perforation-forming structures 1030 (i.e. offset angle D) will correspondingly be less than, or greater than the predefined angle of inclination (i.e., angle C) by a corresponding amount. It is desired to have this inverse amount of the offset angle D match the angular deviation of angle C. Exact matching is, however, not required to reduce the effects of thermal creep. As such, the degree of biasing or offset of the perforation-forming structures 1030 relative to the transverse reference line is arranged to inhibit or prevent the impact of thermal creep causing the perforations to assume a skewed orientation that may result in a film having tearing characteristics other than desired.

The perforation-forming structures 1030 may be etched wells 1030. Such a backing roll 1000 with such etched wells 1030 may be available from Custom Etch Rolls, Inc. of New Castle PA. In an exemplary embodiment, the backing roll 1000 may be plated with a 0.5 mm of copper of 220 Vickers hardness. The illustrated pattern of etched wells, in this embodiment, is a biased pattern that was etched to a depth of 0.23 mm using techniques known in the art. It will be understood, that the present invention contemplates using film supporting apparatus other than backing rolls. For example, the film supporting apparatus may be other equivalent film supporting and conveying devices, such as conveying belts (not shown) or the like.

After etching, the backing roll surface 1020 may be washed with a suitable acid, polished, plated with about 10 microns of chrome, and then re-polished to a mirror finish (4-8 RMS). Such a backing roll 1000 is mounted in the flame-perforating apparatus of the noted U.S. Pat. No. 7,037,100.

In one example (i.e., sample #1), balanced simultaneously biaxially oriented polypropylene (SBOPP) film was then perforated on a bias pattern backing roll by the method described above. This perforation condition is denoted as Standard in Table 1, below.

In another example, another sample (i.e., sample #2) was run, the BOPP film was perforated on a so-called “dry roll”, that is without the presence of a condensed water film on the backing roll 1000 and using a backing roll held at a temperature of 10° C. (50° F.) described in the last noted patent. The dry roll condition was achieved by blowing all of the condensed water off of the backing roll 1000 with intense jets of air through the applicator 50 directed against the backing roll with the condensation air flow control at maximum. Samples were collected at least 10 minutes after process conditions appeared to stabilize.

The total condensation control air-flow using a condensed layer of water method was 450 l/min (16 cfm) while the total condensation-control air flow at the maximum flow was 1290 l/min (45.5 cfm).

Various perforated films were tested for TD and MD tear by a method similar to the “Pinch Tear” test described in Col. 15 of commonly assigned U.S. Pat. No. 7,138,169 which patent is incorporated herein by reference. In preparing Table 1 infra, approximately seventy-five 8 cm×30 cm portions of perforated film samples were cut so that the 30-cm dimensions was oriented in either the TD or MD. For testing for TD tear or MD tear, respectively. Several small 1-cm—long slits were then made with a razor blade (not shown) along the 8-cm edge of the samples to be tested. These slits provided a site for tear initiation. The samples were then torn in accordance with the Pinch tear test noted above. Samples were judged to “fail” the tear test if the number of adjacent rows of perforations across which the tear propagates is equal to or greater than two.

The results of the tear test are reported as “percent failure.” The “hole angle” is the measurement of angle (A) on the perforated film. The “desired angle” is the angle (B) in FIG. 9 and is 45 degrees.

TABLE 1

| | | | Hole | | |
|---|---|---|---|---|---|
| Sample | Perforation | Perforation | Angle | TD Tear | MD Tear |
| # | Pattern | Condition | (°) | (% failure) | (% failure) |
| 1 | 51° bias | Standard- | 45 | 2 | 0 |
| | pattern | condensation | | | |
| | | control | | | |
| 2 | 51° bias | Condensation | 45 | 2 | 4 |
| | pattern | control air flow | | | |
| | | at maximum | | | |
| 3 | 45° | Standard- | 51 | 15 | 0 |
| | herringbone | condensation | | | |
| | | control | | | |
| 4 | 45° | Condensation | 51 | 13 | 4 |
| | herringbone | control air flow | | | |
| | | at maximum | | | |

As evident from the data, the samples of the BOPP perforated film using a bias-patterned backing tool as noted above has a hole angle or major axis at the desired 45 degrees, thereby generating tear that is straight in both the MD and TD with a minimal number of tear failures. The data also illustrates that a significant advantage arises from the patterning of the present invention in that acceptable tear characteristics can be obtained with a so-called dry backing roll (i.e., with the condensation control air flow at a maximum). As sample #2 indicates, the use of biased pattern instead of the use of controlled water condensation on the backing roll would result in a significant improvement in the robustness of a manufacturing scale perforation process. The data of sample #2 is to be compared to the data generated for sample #4, wherein the biasing of the present invention was not utilized with a dry backing roll.

The data also illustrates that a significant advantage arises from the biased patterning of the present invention in that acceptable tear characteristics can be obtained even when a known standard condensation control approach, such as described in the noted U.S. Pat. No. 7,037,100, (i.e., with a water condensation control procedure and apparatus) is utilized. As sample #1 indicates the use of a biased pattern even with a controlled water condensation process on the backing roll would result in a significant improvement compared to the sample #3 wherein a biased pattern was not used.

The present disclosure envisions correcting for any angular offset of the perforation-forming structures from a desired angle of inclination. More typical offsets may range from about 1-15 degrees. This angular offset may either be greater than or less than the desired angle, which in the exemplary embodiment is 45 degrees. Other even more typical ranges for the angular offset may be about least 6-10 degrees greater than or less than the 45 degrees. In one exemplary embodiment as described above, the angular offset of the perforation-forming structures was 6 degrees.

FIG. 11 illustrates BOPP perforated film 1100 having a plurality of perforations 1102 which will have the characteristics of sample #2 of the above Table 1 after the flame-perforating process utilizing the backing roll 1000. As noted, for sample #2 a dry backing roll was used. It will be observed that the resulting perforations 1102 in the BOPP perforated film 1100 have a major axis 1108 coincident with line 1110 that has an angle of inclination that is 45 degrees relative to the transverse reference line 1112. Accordingly, there is provided a film having comparable tearing in both the MD and the TD.

The films described herein are suited for many adhesive tape backing applications, such as described above in regard to FIG. 7. The presence of a top film over the perforation pattern can provide an appearance similar to a poly-coated cloth-based tape backing in certain embodiments. Such an appearance, combined with the tensile and tear characteristics, makes the film useful as a backing for duct tape, gaffer's tape, or the like. Because the backing is conformable, it is also useful as a masking tape backing. It will be appreciated that additional or alternating layers can be used to create the tape.

According to the present disclosure methods, systems, and apparatus are provided for making film having controlled tear characteristics of films, such as flame-perforated films. Aspects of the present disclosure implement being able to easily and reliably perforate film during a flame-perforating process, such that skewing of perforation orientations that are due to thermal creep are minimized or eliminated. Aspects of the present disclosure implement being able to provide tear characteristics wherein polymeric films, such as flame-perforated polymeric films, have comparable tear characteristics in both the lengthwise or machine direction (MD), and the crosswise or transverse direction (TD). Aspects of the present disclosure implement being able to correct for positional skewing of perforations in films, such as flame-perforated films, by thermal creep. Aspects of the present disclosure further include being able to, in a low cost manner, offset the impact of thermal creep skewing the orientations of perforations in film. Aspects of the present disclosure implement further being able to offset the impact of thermal creep skewing the orientation of perforations formed in the film in a manner that lessens the need for adhesion created by a water film, or the relatively expensive and complex water film control methods and mechanisms used during the actual process. Aspects of the present disclosure implement the ability to increase web tensions during the process so as to enable commercial processing of films requiring relatively high tension forces without being affected by thermal creep. According to the present disclosure prior needs are being satisfied such that the true potential for perforating films providing enhanced tear characteristics can be fully achieved, especially in a simple, reliable, and less costly manner.

The aspects described herein are merely a few of the several that can be achieved by using the disclosure. The foregoing descriptions thereof do not suggest that the disclosure must only be utilized in a specific manner to attain the foregoing aspects.

The above embodiments have been described as being accomplished in a particular sequence, it will be appreciated that such sequences of the operations may change and still remain within the scope of the disclosure.

This disclosure may take on various modifications and alterations without departing from the spirit and scope. Accordingly, this disclosure is not limited to the above-described embodiments, but is to be controlled by limitations set forth in the following claims and any equivalents thereof

What is claimed is:

1. A method of correcting for positional skewing of perforations from a predefined angle of inclination relative to a generally transverse reference line of flame-perforated film produced by a flame-perforating process under a first set of conditions, the method comprising: determining the degree of angular deviation of the major axis of each of the one or more perforations in the flame-perforated film from the predefined angle of inclination; and forming one or more perforation-forming structures in a film supporting structure adapted for use in a subsequent flame-perforating process using the first set of conditions, wherein each of the perforation-forming structures has a major axis being angularly offset to the predefined angle of inclination by an inverse amount related to the angular deviation of the one or more corresponding perforations of the previously flame-perforated film.

2. The method of claim 1, wherein the inverse amount of angular offset generally matches the angular deviation.

3. A film-supporting apparatus adapted to form perforations in film supported thereon during a flame-perforating process, wherein each of the formed perforations has a major axis positioned at a predefined angle of inclination relative to a generally transverse reference line, the apparatus comprises: a body having a film supporting surface, and one or more perforation-forming structures positioned on the film supporting surface, wherein each perforation-forming structure has a major axis angularly offset from the predefined angle of inclination of the major axis of each corresponding formed perforation by a predetermined amount.

4. The apparatus of claim 3, wherein the predetermined amount is determined by the method of claim 1.

5. The apparatus of claim 3, wherein the body is a roller and the one or more perforation-forming structures define a pattern for imparting a corresponding pattern of perforations in the flame-perforated film, whereby the pattern of perforation-forming structures controls tear characteristics of the flame-perforated film.

6. The apparatus of claim 5, wherein the pattern is formed so as to produce substantially comparable transverse direction and machine direction tear characteristics.

7. A roller adapted for forming perforations in flame-perforated film, the roller comprises: a body having a film supporting surface; and one or more perforation-forming structures on the film supporting surface and positioned to form corresponding perforations in the film during a flame-perforating process, such that each formed perforation has a major axis at about 45 degrees relative to a generally transverse reference line to the film, wherein each perforation-forming structure has a major axis positioned at a predefined angular offset from the 45 degrees.

8. The roller of claim 7, wherein the predefined angular offset is in a range of about at least 1-15 degrees greater than or less than the 45 degrees.

9. The roller of claim 8, wherein the predefined angular offset is in a range of about at least 6-10 degrees greater than or less than the 45 degrees.

10. The roller of claim 9, wherein the predefined angular offset is about 6 degrees greater than or less than the 45 degrees.

11. A roller adapted for use in a flame-perforating apparatus for perforating film, the roller comprises: a body; a film supporting surface on the body adapted to support and convey film to be perforated; and one or more perforation-forming structures on the supporting surface, each of which has a major axis having an angular orientation that is angularly offset to a predefined angle of inclination that is established relative to a generally transverse reference line across the film to be supported, wherein the angular offset is inversely related to a predefined angular deviation of one or more corresponding skewed perforations formed in previous flame-perforated film that relate to film to be flame-perforated, the film supporting surface thus configured forms perforations in the film to be flame-perforated during a flame-perforating process that offsets the impact of thermal creep skewing the resulting one or more perforations, such that the major axis of each of the resulting one or more formed perforations is generally coincident with the predefined angle of inclination.

12. A method of controlling tear characteristics of film, comprising: providing a polymeric film to be flame-perforated; providing a flame supporting apparatus that includes a film supporting surface having one or more perforation-forming structures, each of the one or more perforation-forming structures has a major axis with an angular orientation that is angularly offset to a predefined angle of inclination established relative to a generally transverse reference line of the film to be supported by the film supporting surface, wherein the angular offset is by an amount that is inversely related to the angular deviation relative to the predefined angle of inclination of one or more corresponding skewed perforations formed in previous flame-perforated film; and applying heat and tension forces to the film as it is advanced by the film supporting apparatus to form resulting perforations in the film; such that the film supporting surface thus configured forms perforations in the film supported thereby that offset the impact of thermal creep skewing the one or more resulting perforations, whereby the major axis of each of the resulting one or more perforations is generally coincident with the predefined angle of inclination.

13. The method of claim 12, further comprising: generating a film of water during a flame-perforating process for adhering the film to the film-supporting surface.

14. A method for use in an apparatus for flame-perforating a film, wherein the apparatus has a film-supporting apparatus as set forth in claim 3, the method of obtaining perforations during a flame-perforating process comprising: using the film-supporting apparatus during a flame-perforating process such that the formed perforations have a major axis at a predefined angle of inclination relative to a generally transverse reference line across the film.

15. A flame-perforated film made according to the process of claim 12.

16. A flame-perforated film comprising: first and second major surfaces; one or more perforations formed in at least one of the first and second major surfaces, wherein the one or more perforations in the film is formed by providing a film supporting apparatus that includes a film supporting surface having one or more perforation-forming structures thereon, each of the perforation-forming structures has a major axis with an angular orientation that is angularly offset to a predefined angle of inclination established relative to a generally transverse reference line of film to be supported by the film supporting surface, wherein the angular offset is by an amount that is inversely related to the angular deviation relative to the predefined angle of inclination of one or more corresponding skewed perforations formed in previous flame-perforated film; and applying heat and tension forces to the film as it is advanced by the film supporting apparatus such that the film supporting surface thus configured forms perforations in the film supported thereby during a flame-perforating process that offsets the impact of thermal creep skewing the resulting one or more perforations, such that the major axis of each of the resulting one or more formed perforations is generally coincident with the predefined angle of inclination.

17. The film of claim 16, wherein the one or more perforation-forming structures in the film supporting surface define a pattern for imparting a corresponding pattern of perforations in the flame-perforated film, whereby the pattern of perforation-forming structures controls tear characteristics of the flame-perforated film.

18. The film of claim 17, wherein the patterns are formed so as to produce substantially comparable transverse direction and machine direction tear characteristics.

19. The film of claim 16, wherein the film is comprised of at least a polymeric material substrate, wherein the polymeric material substrate comprises: a construction that permits perforation by flame.

20. The film of claim 19, wherein the polymeric material substrate is from a group that comprises: films, sheets, porous materials, and foams.

21. The film of claim 19, wherein the polymeric material substrate is from a group that comprises: polyolefins, such as polyethylene, polypropylene, polybutylene, polymethylpentene; mixtures of polyolefin polymers and copolymers of olefins; polyolefin copolymers containing olefin segments such as poly(ethylene vinylacetate), poly(ethylene methacrylate) and poly(ethylene acrylic acid); polyesters, such as poly(ethylene terephthalate), poly(butylene phthalate) and poly(ethylene naphthalate); polystyrenes; vinylics such as poly(vinyl chloride), poly(vinylidene dichloride), poly(vinyl alcohol) and poly(vinyl butyral); ether oxide polymers such as poly(ethylene oxide) and poly(methylene oxide); ketone polymers such as polyetheretherketone; polyimides; mixtures thereof, or copolymers thereof.

22. The film of claim 21, wherein the polymeric material substrate is from a group that comprises: simultaneously or sequentially biaxially oriented polypropylene film, and uniaxially oriented polypropylene film.

23. A flame-perforating apparatus for flame-perforating a film; the flame-perforating apparatus comprises: a frame; a first device coupled to the frame for applying heat to the film to form perforations in the film; and a second device coupled to the frame for advancing the film under tension through the apparatus, the second device includes a film supporting apparatus, the flame supporting apparatus includes a body having one or more perforation-forming structures on a film supporting surface thereof, each of the perforation-forming structures has a major axis with angular orientation angularly offset to a predefined angle of inclination established relative to a generally transverse reference line of film to be supported by the film supporting surface, wherein the angular offset is by an amount that is inversely related to the angular deviation relative to the predefined angle of inclination of one or more corresponding skewed perforations formed in previous flame-perforated film.

24. The flame-perforating apparatus of claim 23, wherein there is further included a water condensation control apparatus for controlling the formation of a film of water on the film supporting surface during the flame-perforating process.

25. The flame-perforating apparatus of claim 22, wherein the body is a roller and the one or more perforation-forming structures define a pattern for imparting a corresponding pattern of perforations in the flame-perforated film, whereby the pattern of perforation-forming structures controls tear characteristics of the flame-perforated film.

26. The flame-perforating apparatus of claim 25, wherein the patterns are formed so as to impart substantially comparable transverse direction and machine direction tear characteristics.

27. A system for flame-perforating film, the system comprises: a film including first and second major surfaces; one or more perforations formed in at least one of the first and second major surfaces; and a flame-perforating apparatus for flame-perforating the film; the flame-perforating apparatus including: a first device for applying heat to the film to form perforations in the film; and a second device for advancing the film under tension through the flame-perforating apparatus, the second device includes a film supporting apparatus, the flame supporting apparatus includes a body having one or more perforation-forming structures on a film supporting surface thereof, each of the perforation-forming structures has a major axis with an angular orientation that is angularly offset to a predefined angle of inclination established relative to a generally transverse reference line of film to be supported by the film supporting surface, wherein the angular offset is by an amount that is inversely related to the angular deviation relative to the predefined angle of inclination of one or more corresponding skewed perforations formed in previous flame-perforated film.

28. The system of claim 27 further including a water condensation control apparatus for controlling the formation of a film of water on the film supporting surface during the flame-perforating process.

29. The system of claim 27, wherein the body is a roller and the one or more perforation-forming structures define a pattern for imparting a corresponding pattern of perforations in the flame-perforated film, whereby the pattern of perforation-forming structures controls tear characteristics of the flame-perforated film.

30. The system of claim 29, wherein the patterns are formed so as to produce substantially comparable transverse direction and machine direction tear characteristics.

31. An adhesive tape comprising: a flame-perforated film having first and second major surfaces as constructed according to claim 16; a first film on one of the first and second major surfaces of the flame-perforated film; and a layer of adhesive coated on at least one of the first film and the other of the first and second major surfaces opposed to the surface having the first film thereon.
